(19) United States
(12) Patent Application Publication
(10) Pub. No.: US 2008/0119733 A1
(43) Pub. Date: May 22, 2008

(54) Selectably compounding and displaying breast ultrasound images

(76) Inventors: Wei Zhang, Union City, CA (US); Jiayu Chen, Palo Alto, CA (US)

Correspondence Address: COOPER & DUNHAM, LLP, 1185 AVENUE OF THE AMERICAS, NEW YORK, NY, 10036, US

(21) Appl. No.: 11/603,665

(22) Filed: Nov. 22, 2006

(51) Int. Cl. A61B 8/13

(52) U.S. Cl. 600/443

(57) ABSTRACT

Acquiring, processing and presenting breast ultrasound information to a user is described. Breast tissue is scanned with an ultrasonic transducer array to generate sonographic image information of the tissue in multiple angles using beamsteering techniques. Sonographic image information is stored in a storage system. One or more image selection buttons are provided that allow the viewer to select which angle or compound image should be displayed. In response to user input, sonographic images are displayed in the desired views, thereby facilitating analysis of the patient's breast tissue by the user. The sonographic image information can be processed real-time, in response to and according to user input. Image selection buttons are described which can soft buttons displayed to the user. The appearance of the soft button can change to indicate the type of image being displayed.

### FIELD

This patent specification relates to medical ultrasound imaging. More particularly, this patent specification relates to processing and/or display of breast ultrasound information for breast cancer screening and/or diagnosis purposes.

### BACKGROUND

Volumetric ultrasound scanning of the breast can serve as a complementary modality for breast cancer screening as described, for example, in the commonly assigned US 2003/0007598A1, US 2003/0212327A1, and US 2005/0171430A1, each of which is incorporated by reference herein. Whereas a conventional two-dimensional x-ray mammogram only detects a summation of the x-ray opacity of individual slices of breast tissue over the entire breast, ultrasound can separately detect the sonographic properties of individual slices of breast tissue, and therefore may allow detection of breast lesions where conventional x-ray mammography alone may not be suitable. Another well-known challenge in x-ray mammography practice is found in the case of dense-breasted women, including patients with high content of fibroglandular tissues in their breasts. Because fibroglandular tissues attenuate x-ray energy more than the surrounding fatty tissues, portions of breasts with high fibroglandular tissue content may not be well penetrated by x-rays and thus the resulting mammograms may contain reduced information in areas where fibroglandular tissues reside. Still another issue in conventional x-ray mammography practice relates to difficulty in imaging near the chest wall, because it is difficult to extend these tissues outward onto the compression plates for proper imaging. A substantial number of cancers are known to occur within 3 cm of the chest wall, which can thereby be missed by x-ray mammography.

In addition to being useful as a complementary modality to x-ray mammography, ultrasound mammography could well become a sole breast cancer screening modality for at least some patient groups. For example, it is believed that preventive health care policy will progress toward the adoption of regular breast cancer screening procedures for increasingly younger women, e.g., women under the age of 40, and perhaps even under the age of 30 if there is a family history of cancer. Because younger women generally have denser breasts, the challenges of conventional two-dimensional x-ray mammography are expected to become especially apparent. Even further, because the dangers of x-ray radiation exposure are cumulative over a lifetime, ultrasound mammography could well become a sole breast cancer screening modality for women in these younger age groups. Other demographics indicating higher breast densities among certain groups, regions, or countries may also lead to the increased adoption of breast ultrasound as a sole or adjunctive screening modality for those groups, regions, or countries.

Key advancements in breast ultrasound technology, as have been set forth in the commonly assigned US 2003/0007598A1, US 2003/0212327A1, and US 2005/0171430A1, supra, and related applications, have improved greatly upon traditional real-time “ad hoc” hand-held breast ultrasound methods with respect to image acquisition, clinical workflow, image display, image archiving, temporal comparison, and other aspects of the breast ultrasound process. In one or more of these advancements, a three-dimensional volume of breast data is acquired by compressing the breast with a taut membranous compressive surface and mechanically sweeping an ultrasound transducer array thereacross to scan the immobilized breast. The three dimensional volume is then stored and can be advantageously used for a variety of subsequent purposes, including display to a physician at a review workstation for analysis, CAD processing, archiving, and subsequent retrieval for temporal comparison.

However, when three-dimensional breast ultrasound data is acquired, processed, and stored according to one or more of the above-described advancements, one problem does arise that is not excessively problematic in real-time “ad hoc” handheld scanning, and relates to shadowing. Shadowing can result from particularly high local tissue attenuation variations in the path of the acoustic waves, as may be caused by ligaments, cysts, or anomalous masses or lesions. Shadows are then cast on the “underlying” tissue positioned further down the acoustic path, making it more difficult to see in the resultant images.

Shadowing is often not a problem for “ad hoc” handheld breast ultrasound, for both technological and practical reasons. Technologically, most ultrasound systems provide for angular compounding, which involves compounding multiple images taken at different angles. Beamsteering techniques are used with an array type ultrasonic transducer to generate multiple partially overlapping component image frames from substantially independent spatial directions or angles. The component frames are combined into a compound image by different techniques including summations, averaging, peak detection, or other combinational means. Examples of spatial compounding from different angular viewpoints can be found in U.S. Pat. No. 6,117,081 (Jago et. al.), U.S. Pat. No. 6,126,598 (Entrekin et. al.), U.S. Pat. No. 6,126,599 (Jago et. al.), and U.S. Pat. No. 6,135,956 (Schmiesing et. al.), each of which are incorporated by reference herein. Other advantages of multi-angle compounding include reducing speckle effects and edge enhancements.

Moreover, even when these technological solutions do not fully resolve a particular shadowing problem, the “ad hoc” handheld process also has the practical advantage of real-time interactivity. In particular, the physician can just manipulate the probe at various angles, while watching the display in real time, until the shadowing is eliminated for the tissue of interest.

However, for the non-real time case (i.e. prior breast ultrasound volume acquisition and subsequent display/processing), although the above-described technological solutions are available and used at the time of image volume acquisition, there is still the issue that real-time interactivity and probe adjustment is not available. Moreover, especially when using larger probe sizes, such as is desirable in volumetric breast ultrasound acquisition, multi-angle compounding can bring about blurring problems.

It would be desirable to provide volumetric breast ultrasound acquisition, processing, and display in a manner that addresses and/or obviates one or more of the above shadowing issues. Other issues and solutions would be apparent to one skilled in the art in view of the present disclosure.

### SUMMARY

A method for acquiring, processing and presenting breast ultrasound information to a user is provided. At least a portion of a patient's breast tissue is scanned with an ultrasonic transducer array to generate sonographic image information of the tissue in a first angle and a second angle with respect to the transducer array. Sonographic image information of the tissue in the first angle and the second angle is stored in a storage system. One or more image selection buttons are provided that allow the user to select whether displayed sonographic images are in the first angle, the second angle, or a compound of at least the first and second angles. User input is received that is indicative of the user's selection with respect to the displayed images. In response to the received user input, sonographic images are displayed in the first angle, the second angle, or a compound of at least the first and second angles, thereby facilitating analysis of the patient's breast tissue by the user. The sonographic image information is preferably processed real-time at the display station in response to, and according to, the received user input. The image selection buttons are preferably soft buttons displayed to the user as one or more of the following: icons, symbols, radio buttons, and words. The appearance of the soft button preferably changes to indicate the type of image being displayed.

A system for acquiring, processing and presenting breast ultrasound information to a user is also provided. An ultrasound image acquisition device has at least one ultrasonic transducer array providing sonographic information of a patient's breast in a first angle and a second angle with respect to the transducer array. An image processing and display station is coupled with the image acquisition device to receive the sonographic information. The display station is configured to display sonographic images and one or more image selection buttons that allow the user to select whether the displayed sonographic images are in the first angle, the second angle, or a compound image of at least the first and second angles. A user input device is adapted to allow the user to select the one or more image selection buttons. The display station also includes a processing system in communication with the display device and the user input device. The processing system is configured to receive the user's selection of the image selection buttons, and in response display sonographic images in the first angle, the second angle, or the compound image of at least the first and second angles.

### BRIEF DESCRIPTION OF THE DRAWINGS

FIG. 1 illustrates a breast cancer screening and/or diagnosis system according to preferred embodiments;

FIG. 2 shows a block diagram of components of an ultrasound imaging system in accordance with preferred embodiments;

FIG. 3 illustrates further detail of a breast ultrasound interface display according to preferred embodiments;

FIG. 4 illustrates a closer view of the menu bar area 128 comprising a variety of controls and information displays relating to the image area 126 on display 130;

FIG. 5 illustrates an alternative button arrangement allowing viewer control over beamsteering angles and compounding, according to a preferred embodiment;

FIG. 6 illustrates another alternative button arrangement allowing viewer control over beamsteering angles and compounding, according to another embodiment;

FIG. 7 illustrates another alternative button arrangement allowing viewer control over beamsteering angles and computing, according to other embodiments;

FIGS. 8a-d illustrate the approximate effects of planar images with various types of beamsteering and compounding; and

FIG. 9 is a flow chart showing steps of acquiring, processing and presenting breast ultrasound information to a user according to certain preferred embodiments.

### DETAILED DESCRIPTION

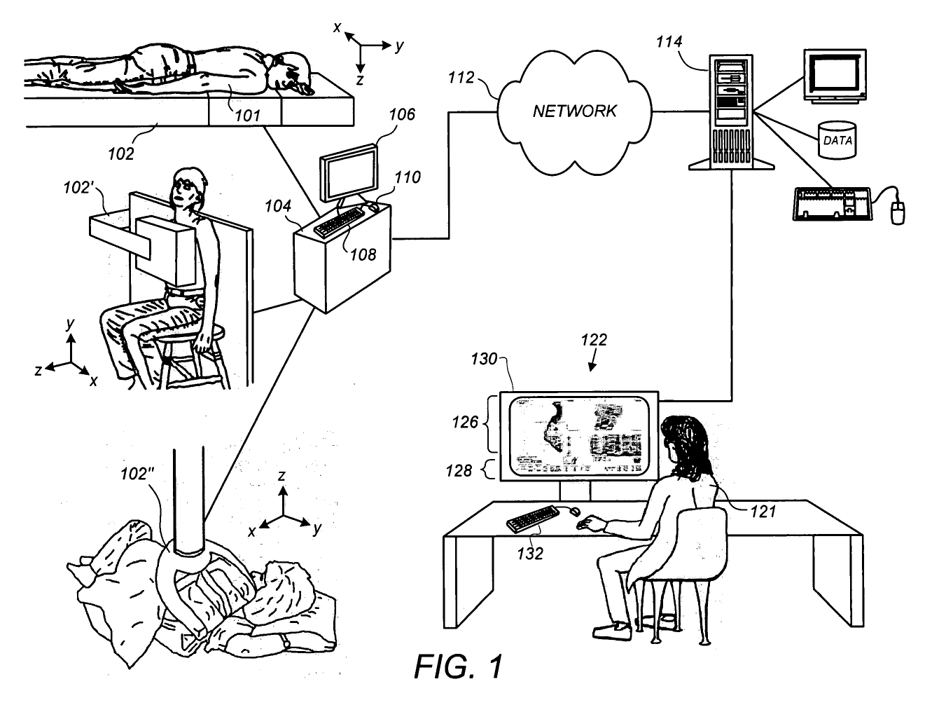

FIG. 1 illustrates a breast cancer screening and/or diagnosis system according to preferred embodiments. The breast of a patient 101 is ultrasonically scanned by an automated scanning apparatus while the patient is in a prone position (device 102), an upright position (device 102′), a supine position (device 102″) or other positions (not shown). By reducing the required ultrasonic penetration depth to the chest wall, scanning of a chestwardly compressed breast can occur at higher frequencies, e.g., 10-20 MHz, which can yield very high resolution images sufficient to facilitate detection of microcalcifications or other structures on the order of 1 mm near the chest wall. However, it is to be appreciated that the scope of the preferred embodiments is not limited to a chestwardly-compressed scenario, with breast ultrasound information processing and display according to the preferred embodiments being generally useful with any scanning system from which a three-dimensional volumetric representation of a sonographic property of the breast can be derived.

Breast scans are obtained under the control of a scanning engine and workstation 104 including, for example, a monitor 106, keyboard 108, a mouse 110, and a scanning engine (not shown). During or after the scanning process, the ultrasound scan data is provided across a computer network 112 to an ultrasound server 114 that processes and generates display information according to the functionalities described herein. The ultrasound server 114 may perform other HIS/RIS (hospital information system/radiology information system) activities such as archiving, scheduling, etc. It is to be appreciated that the processing of the ultrasound scan data may be performed by any of a variety of different computing devices coupled to the computer network 112 in various combinations without departing from the scope of the preferred embodiments.

According to a preferred embodiment, a viewing workstation 122 is provided that displays images to a clinician 121. As used herein, the term “clinician” generically refers to a medical professional, such as a radiologist, or other person that analyzes medical images and makes clinical determinations therefrom, it being understood that such person might be titled differently, or might have varying qualifications, depending on the country or locality of their particular medical environment. Viewing workstation 122 also includes user input devices 132 which ordinarily comprises a keyboard and mouse or other pointing device. The input devices 132 can also include a touch screen incorporated into display 130. High resolution display 130 is preferably used to display images and provide interactive feedback to clinician 121. Display 130 may consist of multiple monitors or a display unit. Shown on display 130 is image area 126 and a menu bar area 128.

In another preferred embodiment, viewing station 122 includes it own separate image processor and memory for processing and displaying in real time, images in response to input from clinician 121.

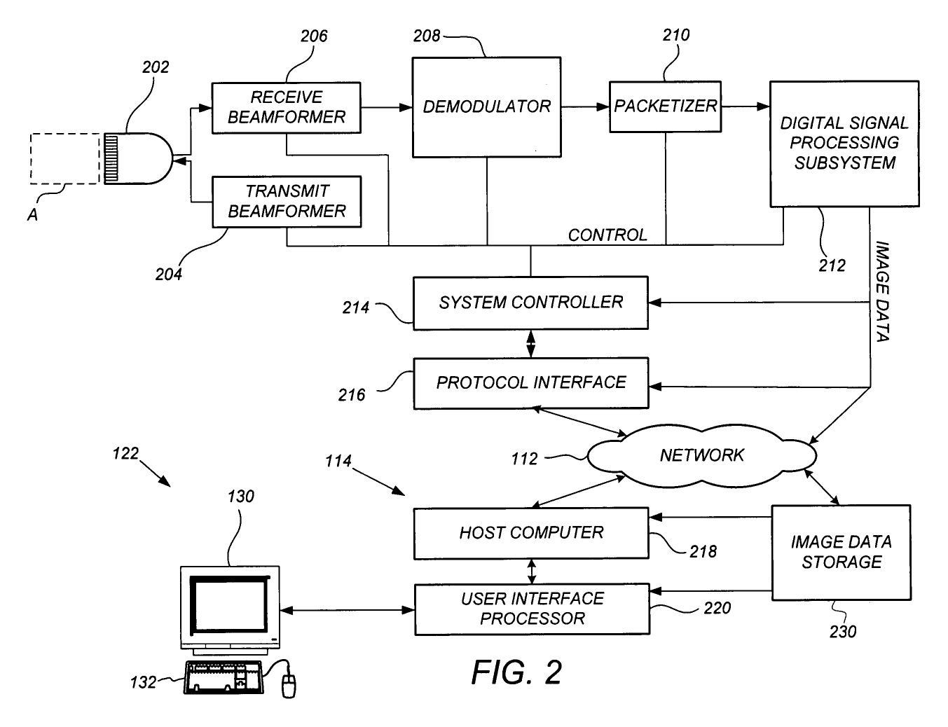

FIG. 2 shows a block diagram of components of an ultrasound imaging system in accordance with preferred embodiments. Transducer 202 comprises an array of transducer elements that transmits focused acoustic signals into a target responsive to signals generated by the transmit beamformer 204. In a preferred embodiment, transducer 202 transmits acoustic pulses into an area A that is fixed relative to the transducer. According to preferred embodiments, angular beamsteering, in which component frames are taken at different angles relative to the transducer, is used.

Responsive to control signals and parameters received from system controller 214, transmit beamformer 204 generates signals that are converted into acoustic interrogation signals by transducer 202 and introduced into the target human tissue A. Transducer 202 also receives acoustic echoes from the target and converts them into signals for forwarding to receive beamformer 206. Receive beamformer 206 receives the signals and converts them into a single-channel RF signal. Demodulator 208 receives the single-channel RF signal and generates component frames therefrom, which are then packetized by packetizer 210 and fed to DSP subsystem 212. In accordance with control signals and compounding weights received from system controller 214, DSP subsystem 212 is able to continuously generate compound output images by compounding component frames. The output image data is transferred to protocol interface 216, but may optionally be further processed by system controller 214. According to a preferred embodiment, DSP subsystem 212 does not perform compounding of the image frames and the uncompounded image data is transferred directly to controller 214, protocol interface 216, and/or image data storage 230, optionally via network 112. The image frame data are then transferred via network 112 to host computer 218 which is preferably part of ultrasound server 114. Image data storage 230 is also preferably part of ultrasound server 114.

According to preferred embodiments, image data storage 230 contains un-compounded image data. In response to user input received from input devices 132 in viewing station 122, the image data is processed by host computer 218 and displayed to the user at viewing station 122 via display 130. As described more fully below, if the user indicates a preference to view a particular original non-compounded image, the non-compounded images are displayed on display 130 in real time. If the user indicates a preference to view a compound image, host computer 218 compounds the image according to the user's preference and displays the compounded image on display 130 in real-time.

According to an alternative embodiment, as described above the image compounding can be performed by DSP subsystem 212 and stored on image data storage 230. In this embodiment, in the case when the user indicates a preference to view a compounded image, the host computer (or user interface processor directly) transfers and displays the appropriate stored compounded image.

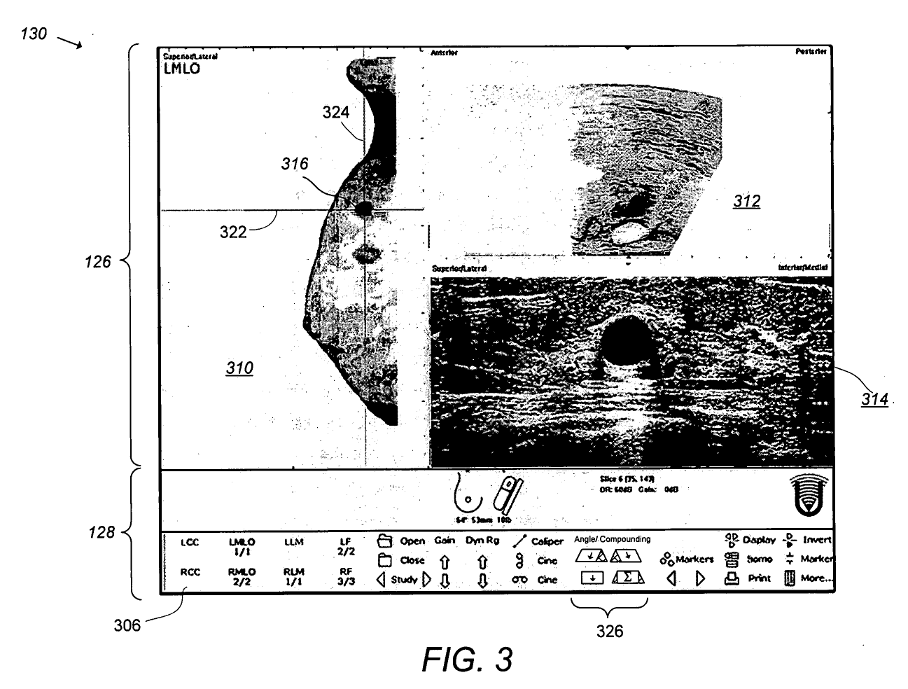

FIG. 3 illustrates further detail of a breast ultrasound interface display according to preferred embodiments. Display 130 generally comprising an image area 126 and a menu bar 128. In the particular display of FIG. 3, the left side of image area 126 illustrates a single thick-slice image 310 (shown in inverted grayscale). On image 310 is shown a cursor 316. FIG. 3 further illustrates planar ultrasound images 312 (shown in inverted grayscale) and 314 corresponding respectively to the plane indicators 322 and 324, which intersect at the current cursor location on image 310. Note that display 130 is preferably part of viewing workstation 122 of FIG. 1, supra. The display 130 can be used as part of a multi-modality PACS workstation, as a stand-alone device, and/or in conjunction with an x-ray mammography softcopy or hardcopy (i.e., lightbox) viewing station. As described more fully below, menu bar area 128 includes angle/compounding buttons 326 through which the user can indicate weather the planar images shown on display 130 are angle beamsteered images at various angles, or compound images of the beamsteered image data.

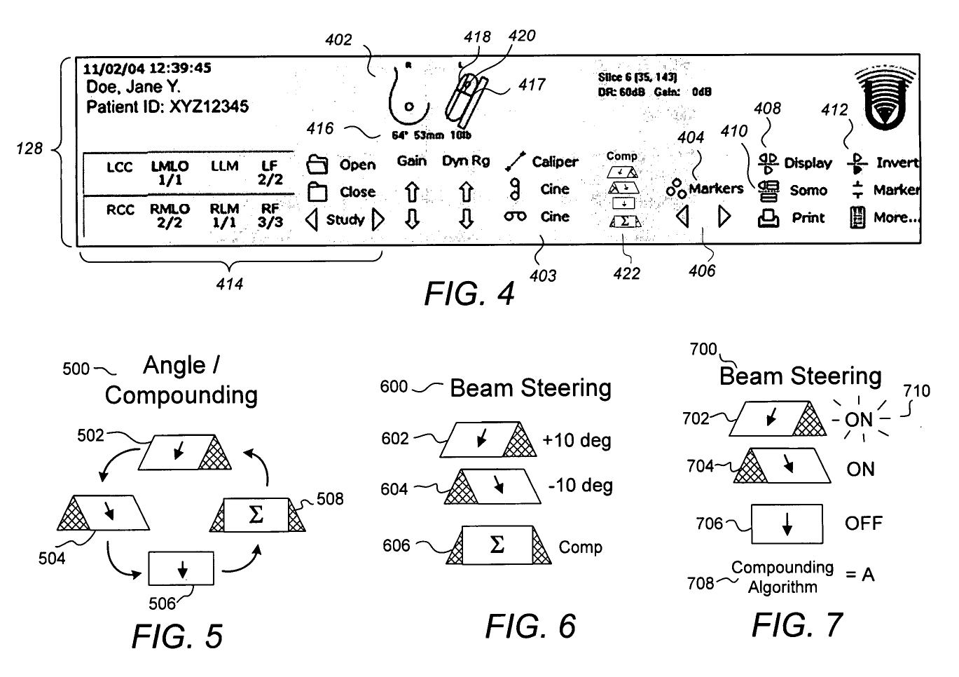

FIG. 4 illustrates a closer view of the menu bar area 128 comprising a variety of controls and information displays relating to the image area 126 on display 130. Menu bar area 128 comprises a body marker icon 402, cine control (soft) buttons 403, a marker display button 404, marker navigation buttons 406, a bilateral comparison control button 408, a sonogram button 410, an invert button 412, and a variety of file control buttons 414. For one preferred embodiment, functionality of the various buttons in menu bar area 128 is similar to that described in U.S. patent application Ser. No. 10/997,293, pub. no. US2005/0171430A1, supra. According to preferred embodiments also provided in menu area 128 is beamsteering angle/compounding buttons 422. Beamsteering angle/compounding buttons 422 allow the viewer to select the type of image displayed in the display area. In particular using buttons 422, the viewer can choose between various beamsteering angles, such as +10 degrees, −10 degrees, or zero degrees (i.e. perpendicular to the ultrasound probe), as well as a compounded image of the beamsteered images. Buttons 422 are designed to symbolically represent different beamsteering angles and compounding in a way that is intuitive to the viewer without causing undue clutter to menu area 128.

As used herein, the term “button” includes many alternative techniques including displayed icons, symbols, shapes such as radio buttons, letters, words and short phrases of text. The appearance of the buttons may change in color, brightness, shading, texture, and blinking or flashing appearance, depending upon the particular design. The term “button” as used herein also refers to both “soft” buttons being displayed and activated by user input device such as a pointing device or touch screen, as well as conventional hardware buttons, switches, levers and the like. Although several different types of buttons are described in the embodiments herein, many other techniques of providing buttons are possible.

FIG. 5 illustrates an alternative button arrangement allowing viewer control over beamsteering angles and compounding, according to a preferred embodiment. In FIG. 5, text label 500 and a single button is displayed, with the button symbolically representing the type of image or images being displayed. Further, the single button appearance and the type of images displayed cycles between various states in response to mouse clicks from the user. In one example, symbol button 502 indicates that images corresponding to a +10 degree beamsteering angle are being displayed the display area. A mouse click on the symbol button 502 causes the displayed image to change quickly to a −10 degree beamsteering angle and the symbol button 504 is displayed in the menu bar. Another mouse click causes zero degree beamsteering images to be displayed in the display area and symbol button 506 to be displayed in the menu bar. Another mouse click causes compounded images to appear in the display area and symbol button 508 to appear in the menu bar. Yet another mouse click returns to the state where symbol button 502 appears in the menu bar and the associated images are displayed.

FIG. 6 illustrates another alternative button arrangement allowing viewer control over beamsteering angles and compounding, according to another embodiment. In FIG. 6, text label 600 and symbol buttons 602, 604 and 606 represent +10 degree beamsteering, −10 degree beamsteering and compounding, respectively. In this embodiment, the viewer simply clicks on the appropriate button to cause the appropriate type of images to be quickly displayed in the display area. Although beamsteering angles of 0, +10 degrees and −10 degrees have been used in the examples described above, other beamsteering angles could also be used with the embodiment described herein.

FIG. 7 illustrates another alternative button arrangement allowing viewer control over beamsteering angles and compounding, according to other embodiments. In FIG. 7 each button is turned on or off individually. In particular text label 700 indicates the general functionality of the buttons on the menu bar. Buttons 702 and 704 each toggle between on and off, and each represents beamsteering in the directions shown. Likewise, button 706 toggles between on and off and represents a zero degree beamsteering angle (i.e. perpendicular to the ultrasound array in the probe). According to a preferred embodiment, text labels such as text label 710 indicate “ON” or “OFF” and are used to indicate the current position of buttons 702, 704 and 706. If only one of buttons 702, 704 and 706 are “ON” then that beamsteering angle is used for the display images. If two or more of buttons 702, 704 and 706 are “ON”, then the appropriate image data is compounded and displayed. If none of the buttons 702, 704 and 706 are “ON” then there is a default beamsteering angle, such as the zero degree angle which is displayed, and the appropriate button (for example button 706 if zero degree is the default) is automatically switched on. Alternatively, when the last “ON” button of buttons 702, 704 and 706 is switched “OFF” then the least recently deselected (i.e. least recently toggled to the “OFF” position) is automatically turned “ON” and that beamsteering angle image is displayed. Finally, a compounding algorithm button 708 is provided for the user to select the type of compounding algorithm. If there are more than two possible compounding algorithms (for example, summations, averaging and peak detection), then button 708 preferably cycles through each of the different choices.

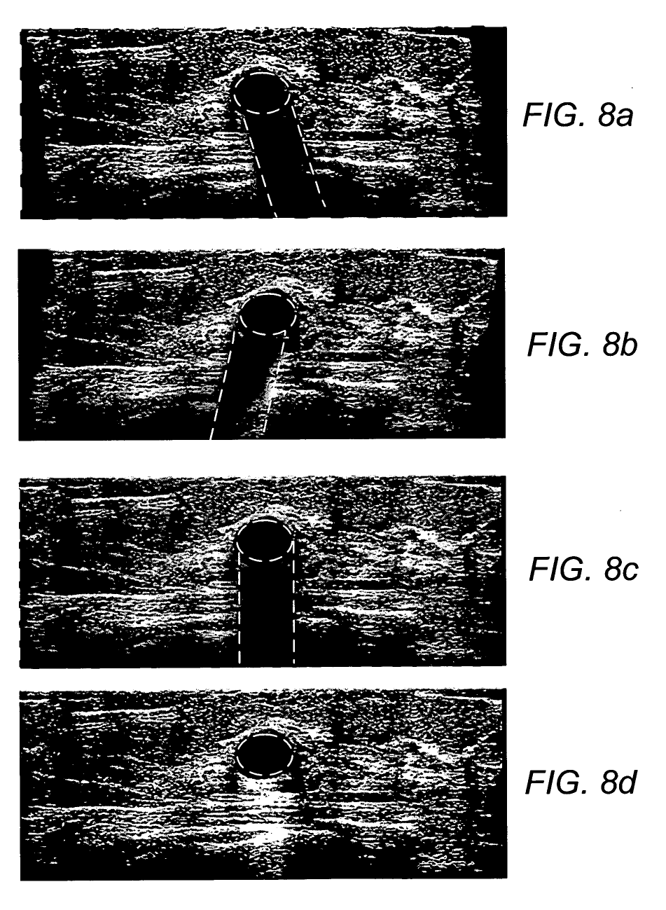

FIGS. 8a-d illustrate the approximate effects of planar images with various types of beamsteering and compounding. In all of the FIGS. 8a-d an oval shaped mass is depicted by the dashed oval line. FIG. 8a illustrates the effect of a beamsteering angle to the right side of the image. The mass causes a shadow area depicted by the white dashed lines. FIG. 8b illustrates the effect of a beamsteering angle to the left side of the image with the associates shadow area again shown in dashed lines. FIG. 8c illustrates the effect of a zero degree beamsteering and the associated shadow is directly beneath the mass. Finally, FIG. 8d illustrates a potential benefit of compounding the beamsteering images to alleviate the effects of the shadows caused by the mass.

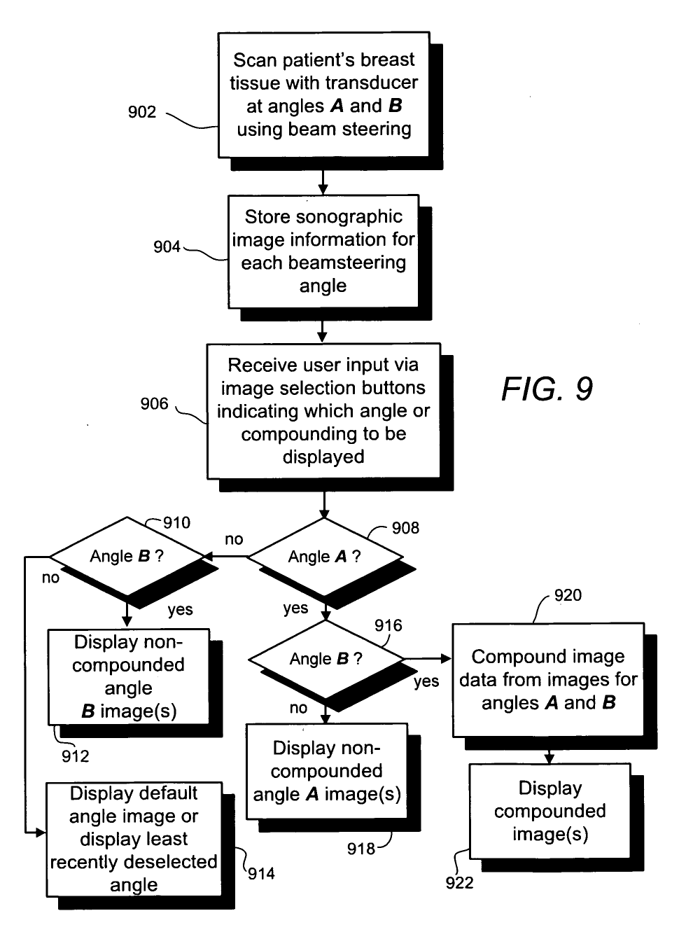

FIG. 9 is a flow chart showing steps of acquiring, processing and presenting breast ultrasound information to a user according to certain preferred embodiments. In step 902, the patient's breast tissue is scanned with an ultrasound probe having an ultrasound array. Beamsteering is preferably used to generate sonographic image data in multiple angles with respect to the ultrasound array. In step 902, two exemplary angles A and B are used. In step 904, the sonographic image information is stored in a storage system. In step 906 the user's input is received via image selection buttons that are displayed or otherwise provided to the user. The buttons preferably represent symbolically the different beamsteering angles captured and stored in steps 902 and 904.

The decision tree formed by decision boxes 908, 910 and 916 are used to determine the state of the user's current selection for type of image to be displayed. If angle A is selected but angle B is not selected, then in step 918 the non-compounded image or images corresponding to angle A are displayed. Note that in many situations, it is desirable to display more than one image of the breast at one time (for example, the two planar images 312 and 324 in FIG. 3). In such cases, it is preferable that both of the images are displayed according to the user's selection. That is, both planar images are displayed using angle A, in the case of step 918. If Angle A is not selected and Angle B is selected, then the stored non-compounded image or images corresponding to angle B are displayed in step 912. If both angle A and angle B are selected by the user, than in step 920 the stored image data from angles A and B are processed to generate a compound image. In step 922 the compound image is displayed. If neither angle A nor angle B is selected, then in step 914 either a default angle is displayed, or in the case of only two different angles, deselecting one angle will cause the other angle to be automatically selected. In the general case where there are more than two angles, then the least recently deselected angle can be automatically selected and displayed.

While the invention has been described in conjunction with the exemplary embodiments described above, many equivalent modifications and variations will be apparent to those skilled in the art when given this disclosure. By way of example, although one or more of the above preferred embodiments is described in terms of exemplary beamsteering angles of +/−10 degrees, it is to be appreciated that beamsteering by other angles, even substantially more extreme angles such as +/−45 degrees or +/−60 degrees (where technically possible) are not outside the scope of the preferred embodiments. Also, beamsteering at more than two or three angles can be used, with provisions allowing the user to select for display and/or compounding images corresponding to different angles. Accordingly, the exemplary embodiments set forth above are considered to be illustrative and not limiting. Various changes to the described embodiments may be made without departing from the spirit and scope of the invention. Reference to the details of the embodiments described are not intended to limit the scope of the invention, which is limited only by the scope of the claims set forth below.

What is claimed is:

1. A method for acquiring, processing and presenting breast ultrasound information to a user comprising the steps of:
scanning at least a portion of a patient's breast tissue with an ultrasonic transducer array to generate sonographic image information of the tissue in a first angle and a second angle with respect to the transducer array;
storing said sonographic image information of the tissue in the first angle and the second angle in a storage system;
providing one or more image selection buttons that allow the user to select whether displayed sonographic images correspond to the first angle, the second angle, or are a compound of sonographic information in at least the first and second angles;
receiving user input indicative of the user's selection with respect to the displayed images; and
in response to the received user input, displaying sonographic images corresponding to the first angle, corresponding to the second angle, or which are a compound of sonographic information in at least the first and second angles, thereby facilitating analysis of the patient's breast tissue by the user.

2. A method as in claim 1 further comprising the step of processing, in response to and according to the received user input, the sonographic image information stored in the storage system according to the received user input.

3. A method as in claim 1 in which said step of scanning includes generating sonographic information in a third angle with respect to the transducer array.

4. A method as in claim 1 in which the third angle is perpendicular to the axis of the transducer array.

5. A method as in claim 1 in which said step of providing one or more image selection buttons is selectable according to the preference of the user.

6. A method as in claim 1 in which the one or more image selection buttons comprises a soft button displayed to the user as one or more of the following: icons, symbols, radio buttons, and words.

7. A method as in claim 6 in which the appearance of the soft button changes to indicate the type of image being displayed.

8. A method as in claim 6 in which said step of receiving user input includes receiving signals from a touch sensitive screen.

9. A method as in claim 6 in which said step of receiving user input includes receiving signals from a pointing device.

10. A method as in claim 6 in which the one or more image selection buttons further comprise a first soft button symbolically representing the first angle and a second soft button symbolically representing the second angle, each of the first and second soft buttons when selected by the user causes the displaying of sonographic images in the first and second angles respectively to toggle on and off, and when both the first and second soft buttons are set to display that type of image, said step of displaying includes displaying a compound of at least the first and second angles.

11. A method as in claim 1 wherein an algorithm used in said step of displaying the compound of at least the first and second angles is selectable by the user and wherein said step of providing includes providing a compounding algorithm selection button.

12. A system comprising:
an ultrasound image acquisition device having at least one ultrasonic transducer array providing sonographic information of a patient's breast in a first angle and a second angle with respect to the transducer array;
an image processing and display station coupled with the image acquisition device to receive said sonographic information therefrom, said station comprising:
a display device configured to display sonographic images and one or more image selection buttons responsive to a user selection to select whether the displayed sonographic images correspond to the first angle, the second angle, or are compounded from sonographic information in at least the first and second angles;
a user input device adapted to respond to a user selection to select the one or more image selection buttons; and
a processing system in communication with said display device and said user input device, said processing system configured to receive the user's selection of the one or more image selection buttons, and in response display sonographic images that correspond to the first angle, the second angle, or are compounded from sonographic information in at least the first and second angles.

13. A system as in claim 12 in which the processing system generates the compound image in real-time in response to the user's selection.

14. A system as in claim 13 wherein an algorithm used to generate the compound image is selectable by the user and wherein said display device is further configured to display a compounding algorithm selection button.

15. A system as in claim 12 in which the ultrasound image acquisition device is configured and programmed to provide sonographic information in a third angle with respect to the transducer array.

16. A system as in claim 15 in which the third angle is perpendicular to the axis of the transducer array.

17. A system as in claim 12 in which the one or more image selection buttons comprise a soft button displayed to the user as one or more of the following: icons, symbols, radio buttons, and words.

18. A system as in claim 17 in which the appearance of the soft button changes to indicate the type of image being displayed.

19. A system as in claim 12 in which said user input device includes a touch sensitive screen.

20. A system as in claim 12 in which said user input device comprises a pointing device.

21. A system as in claim 12 in which the one or more image selection buttons further comprise a first soft button symbolically representing the first angle and a second soft button symbolically representing the second angle, each of the first and second soft buttons when selected by the user causes the displaying of sonographic images in the first and second angles respectively to toggle on and off, and when both the first and second soft buttons are set to display that type of image, said step of displaying includes displaying a compound of at least the first and second angles.

22. A computer program stored on a tangible medium, comprising:
computer code for scanning at least a portion of a patient's breast tissue with an ultrasonic transducer array to generate sonographic image information of the tissue in a first angle and a second angle with respect to the transducer array;
computer code for providing one or more image selection buttons that respond to a user selection to select whether displayed sonographic images are in the first angle, the second angles, or a compound of at least the first and second angles;
computer code for receiving user input indicative of the user's selection with respect to the displayed images; and
computer code for displaying, in response to the received user input, sonographic images corresponding to the first angle, the second angle, or that are a compound of sonographic information in at least the first and second angles, thereby facilitating analysis of the patient's breast tissue by the user.
